(19) United States
(12) Patent Application Publication
(10) Pub. No.: US 2015/0139950 A1
(43) Pub. Date: May 21, 2015

(54) ALISPORIVIR TO TREAT HEPATITIS C VIRUS INFECTION

(71) Applicant: Claudio Avila, Basel (CH)

(72) Inventor: Claudio Avila, Basel (CH)

(73) Assignee: Novartis AG, Basel (CH)

(21) Appl. No.: 14/540,469

(22) Filed: Nov. 13, 2014

Related U.S. Application Data
(63) Continuation of application No. 14/007,499, filed on Sep. 25, 2013.
(60) Provisional application No. 61/470,152, filed on Mar. 31, 2011.

(51) Int. Cl. A61K 38/12; A61K 38/21; A61K 31/7056; A61K 45/06

(52) U.S. Cl. CPC A61K 38/12; A61K 45/06; A61K 38/212; A61K 31/7056
USPC 424/85.7; 514/4.3; 530/321

(57) ABSTRACT

The disclosure concerns the use of cyclophilin inhibitors in the treatment of chronic Hepatitis C virus infection.

The present disclosure relates to a non-immunosuppressive cyclosporin which binds to cyclophilin, which are cyclophilin inhibitors, in particular to their pharmaceutical use of in the treatment of Hepatitis C virus infection.

The cyclosporins comprise a class of structurally distinctive, cyclic, poly-N-methylated undecapeptides, commonly possessing pharmacological, in particular immunosuppressive, or anti-inflammatory activity. The first of the cyclosporins to be isolated was the naturally occurring fungal metabolite Ciclosporin or Cyclosporine, also known as cyclosporin A (CsA).

Cyclosporins which bind strongly to cyclophilin but are not immunosuppressive have been identified. PCT/EP 2004/009804, WO 2005/021028, or WO 2006/071619 disclose non-immunosuppressive cyclosporins which bind to cyclophilin have also been found to have an inhibitory effect on Hepatitis C virus (HCV). WO 2006/038088, incorporated herein by reference in its entirety, describes methods and compositions for the use of alisporivir in the treatment of HCV. Alisporivir (DEBO25 or Debio-025) is a cyclophilin (Cyp) inhibitor and its mode of action as an anti-HCV agent is via inhibition of host proteins, in particular of cyclophilin A, that are directly involved in HCV replication.

Hepatitis C virus (HCV) is an enveloped single stranded (+) RNA virus that belongs to the separate genus Hepacivirus of the family Flaviviridae. HCV causes acute and chronic liver disease, including chronic hepatitis, cirrhosis, and hepatocellular carcinoma. Worldwide more than 170 million people are chronically infected with HCV and are thus at increased risk of developing serious life-threatening liver disease. Persistent infection by HCV, which has been identified as the major causative agent of non-A, non-B hepatitis has been considered closely related to liver diseases such as chronic hepatitis, liver cirrhosis or hepatocellular carcinoma. The development of these liver diseases is a major public health problem.

The current standard of care in HCV patients consists of a combination of interferon and ribavirin. Treatment duration and ribavirin dose depend on the genotype treated. Sustained viral response (SVR) in patients with genotypes 2 and 3 after standard of care treatment reaches 80-90%, but only 40-50% in patients with genotype 1. Furthermore, side effects are significant and include myalgia, arthralgia, headache, fever, severe depression, leucopenia and haemolytic anaemia. The current standard of care is treatment with pegylated intereferon and ribavirin.

Genetic variation in the region of the interleukine 28B gene (IL28B) encoding for interferon lambda 3 has been shown to be critical for treatment response to standard of care in patients infected with HCV. From the SPNs identified on chromosome 19 near the IL28B gene, the rs12979860 SNP strongly associates with the response to standard of care in patients infected with chronic HCV infection, is located 3 kilobase upstream of the IL28B gene and is a bi-allelic C/T polymorphism. Individuals may carry either 2 copies of the C allele, or 2 copies of the T allele or both T and C alleles, therefore three genotypes may exist: IL28B C/C, IL28B T/T or IL28B T/C. The genotypes IL28B T/T and T/C are hereinafter called IL28B nonC/C genotype.

The patients with IL28B C/C genotype are most likely to have the best treatment outcomes whereas those with the nonC/C genotype have the less favorable response to the treatment with standard of care.

Sustained virological response was achieved by 84% and 32% with genotype IL28B C/C, and IL28B nonC/C, respectively when patients infected with HCV genotype 1 are treated with a direct antiviral telaprevir and standard of care (see Gohl, P., IL28B (Interleukin 28B)-gene polymorphism: impact on natural course and treatment of hepatitis C Bioscientia, 2011).

This clearly showed that a significantly higher proportion of patients with genotype IL28B C/C showed sustained virological response than that of patients with IL28B nonC/C genotypes.

Surprisingly we have found out that cyclophilin inhibitors, in particular alisporivir, can be used effectively in the treatment of HCV of patients with IL28B nonC/C genotype. In particular, we have found that particularly satisfactory treatment results of chronic Hepatitis C virus genotype 1 infection in patient with IL28B nonC/C genotype are obtained when alisporivir is used. Furthermore, we have found that cyclophilin inhibitors, in particular alisporivir, can also be used effectively in the treatment of HCV of patients with IL28B C/C genotype.

The invention further provides alisporivir for use in the treatment or prevention of chronic Hepatitis C virus genotype 1 infections or HCV induced disorders in a patient with IL28B nonC/C genotype, treatment naive.

Accordingly, the present invention provides new anti-HCV treatments using alisporivir, in particular methods of treating hepatitis C virus genotype 1 infection in a patient with IL28B nonC/C genotype comprising administering to the patient alisporivir, during an initial phase in an amount of about 400 to about 600 mg twice a day; followed by administering alisporivir during a second phase in an amount of about 600 to about 800 mg once per day.

Furthermore, the present disclosure provides a method of treatment of chronic Hepatitis C genotype 1 infections comprising first determining the IL28B genotype of the patient and secondly administering alisporivir alone or in combination with standard of care.

### SUMMARY OF THE DISCLOSURE

Further, the following is described:

1.1 A method for preventing or treating chronic Hepatitis C infections or HCV induced disorders in a patient with IL28B nonC/C genotype, comprising administering to said patient alisporivir during an initial phase in an amount of about 400 to about 600 mg twice a day; followed by administering alisporivir during a second phase in an amount of about 600 to about 800 mg once per day.

1.2 A method for inhibiting HCV replication in a patient with IL28B nonC/C genotype, comprising administering alisporivir during an initial phase in an amount of 600 mg twice a day; followed by administering alisporivir during a second phase in an amount of about 600 to about 800 mg once per day.

1.3 A method for preventing the recurrence of HCV infection in a transplant recipient, with IL28B nonC/C genotype comprising administering to said recipient alisporivir during an initial phase in an amount of about 600 mg twice a day; followed by administering alisporivir during a second phase in an amount of about 600 to about 800 mg once per day.

1.4 A method for preventing or treating chronic Hepatitis C infections or HCV induced disorders in a patient with IL28B C/C genotype, comprising administering to said patient alisporivir during an initial phase in an amount of about 400 to about 600 mg twice a day; followed by administering alisporivir during a second phase in an amount of about 600 to about 800 mg once per day.

1.5 A method for inhibiting HCV replication in a patient with IL28B C/C genotype, comprising administering alisporivir during an initial phase in an amount of 600 mg twice a day; followed by administering alisporivir during a second phase in an amount of about 600 to about 800 mg once per day.

1.6 A method for preventing the recurrence of HCV infection in a transplant recipient, with IL28B C/C genotype comprising administering to said recipient alisporivir during an initial phase in an amount of about 600 mg twice a day; followed by administering alisporivir during a second phase in an amount of about 600 to about 800 mg once per day.

2. Use of alisporivir in the preparation of a pharmaceutical composition for use in any method as defined above.

3. Use of alisporivir in the preparation of a medicament for use in any method as defined above.

4. A pharmaceutical composition for use in any method as defined above, comprising alisporivir, together with one or more pharmaceutically acceptable diluents or carriers therefore.

5. A therapeutic regimen comprising administering alisporivir during an initial phase in an amount of about 400 to about 600 mg, twice per day followed by administering alisporivir during a second phase in an amount of about 600 to about 800 mg once per day and wherein alisporivir is administered in combination with standard of care throughout the initial and second phases to a patient with IL28B nonC/C genotype;

6. A method of treatment of chronic Hepatitis C genotype 1 infections comprising

(i) determining the IL28B genotype of the patient

(ii) administering alisporivir during an initial phase in an amount of about 400 to about 600 mg, twice per day followed by administering alisporivir during a second phase in an amount of about 600 to about 800 mg once per day and wherein alisporivir is administered in combination with standard of care throughout the initial and second phases to a patient with IL28B nonC/C genotype;

(iii) administering alisporivir during an initial phase in an amount of about 400 to about 600 mg, twice per day followed by administering alisporivir during a second phase in an amount of about 600 to about 800 mg once per day and wherein alisporivir is administered in combination with standard of care throughout the initial and second phases to a patient with IL28B C/C genotype and wherein the treatment duration is halved compared to the treatment duration of the approved standard of care treatment duration for genotype 1 patients.

7. A package comprising the pharmaceutical composition comprising alisporivir as defined above, in combination with instructions to administer said composition during an initial phase in an amount of about 600 mg twice a day; followed by administering alisporivir during a second phase in an amount of about 600 to about 800 mg once per day.

8. A kit for the treatment of chronic hepatitis C infection.

Also contemplated herein are methods of reducing the HCV RNA in patient comprising administering to the patient: alisporivir, an interferon; and a ribavirin in which alisporivir is administered during an initial phase in an amount of about 400 to about 600 mg twice a day; followed by administering alisporivir during a second phase in an amount of about 600 or about 800 mg once per day.

Additional embodiments of the present invention relate to methods of treating chronic hepatitis C genotype 1 infections in a patient with IL28B nonC/C genotype comprising administering to the patient: alisporivir in combination with standard of care, wherein alisporivir is administered during an initial phase in an amount of about 600 mg twice a day; followed by administering alisporivir during a second phase in an amount of about 600 to about 800 mg once per day.

Additional embodiments of the present invention relate to methods treatment of chronic Hepatitis C genotype 1 infections comprising

(i) determining the IL28B genotype of the patient

(ii) administering alisporivir during an initial phase in an amount of about 400 to about 600 mg, twice per day followed by administering alisporivir during a second phase in an amount of about 600 to about 800 mg once per day and wherein alisporivir is administered in combination with standard of care throughout the initial and second phases to a patient with IL28B nonC/C genotype;

(iii) administering alisporivir during an initial phase in an amount of about 400 to about 600 mg, twice per day followed by administering alisporivir during a second phase in an amount of about 600 to about 800 mg once per day and wherein alisporivir is administered in combination with standard of care throughout the initial and second phases to a patient with IL28B C/C genotype and wherein the treatment duration is halved compared to the treatment duration of the approved standard of care treatment duration for genotype 1 patients.

Also contemplated herein is a pharmaceutical combination comprising a first pharmaceutically acceptable formulation comprising alisporivir, a second pharmaceutically acceptable formulation comprising an interferon and a third pharmaceutically acceptable formulation comprising ribavirin, wherein the first, second and third formulations are packaged in a kit for the treatment of chronic hepatitis C infection.

### Detailed Description of the Disclosure

### BRIEF DESCRIPTION OF THE DRAWINGS

FIG. 1 shows the results of treatment with alisporivir according to methods disclosed herein, in particular corresponding to the treatment arms as disclosed in the Examples, in patients with IL28B C/C genotype.

FIG. 2 shows the results of treatment with alisporivir according to methods disclosed herein, in particular corresponding to the treatment arms as disclosed in the Examples, in patients with IL28B nonC/C genotype.

In the above embodiments and throughout this specification, the standard of care treatment is a treatment that is used to treat Hepatitis C infections. The currently used standard of care treatment involves administration of interferon, in particular pegylated interferon in combination with ribavirin.

In some embodiments, the patient may be coinfected with another virus, e.g., human immunodeficency virus (HIV), or the patient may be a transplant recipient (e.g., a liver transplant recipient)

In the above embodiments and throughout this specification, the initial phase is a period of 3, 4, 5, 6, or 7 days. Preferably the initial phase is a period of at least 3 days, preferred of 7 days.

In the above embodiments and throughout this specification, the second phase is a period of 23, 47 or 71 weeks. Preferably the second phase is a period of 47 weeks.

In the present application, the term “non-responder” is intended to mean a patient or subject who is a non-responder to standard of care treatment for HCV. More specifically, a non-responder to standard of care patient is a patient who has not responded to treatment with standard of care given over a 12 week treatment period. The non-responder to standard of care includes the following subsets of patients—null responders and partial responders.

Typically, a patient who has a “null response” may, for example, be defined as one in whom the HCV-RNA reduction is observed to be less than about 2 log 10 IU/mL (e.g., less than 2 log 10 IU/mL) after 12 weeks of treatment with standard of care.

A patient that has a “partial” response or partial responder is one in whom the HCV-RNA reduction of more than about 2 log 10 IU/mL (e.g., less than 2 log 10 IU/mL) is observed after 12 weeks of treatment with standard of care but the HCV-RNA is still detectable at the end of treatment.

In the present invention, an interferon may be pegylated or non-pegylated and may include interferons such as: Intron-A®, interferon alfa-2b (Schering Corporation, Kenilworth, N.J.); PEG-Intron®, peginteferon alfa-2b (Schering Corporation, Kenilworth, N.J.); Roferon®, recombinant interferon alfa-2a (Hoffmann-La Roche, Nutley, N.J.); Pegasys®, peginterferon alfa-2a (Hoffmann-La Roche, Nutley, N.J.); Berefor®, interferon alfa 2 available (Boehringer Ingelheim Pharmaceutical, Inc., Ridgefield, Conn.); Sumiferon®, a purified blend of natural alpha interferons (Sumitomo, Japan); Wellferon®, lymphoblastoid interferon alpha n1 (GlaxoSmithKline); Infergen®, consensus alpha interferon (InterMune Pharmaceuticals, Inc., Brisbane, Calif. and Amgen, Inc., Newbury Park, Calif.); Alferon®, a mixture of natural alpha interferons (Interferon Sciences, and Purdue Frederick Co., CT); Viraferon®; and combinations of these interferons.

Conjugated interferons that may be used include, for example, Albuferon (Human Genome Science) which is conjugated to human albumin. Interferon conjugated to a water-soluble polymer or polyalkylene oxide homopolymers such as polyethylene glycol (PEG) or polypropylene glycols, polyoxyethylenated polyols, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used. Interferon-polymer conjugates are described in U.S. Pat. No. 4,766,106, U.S. Pat. No. 4,917,888, EPA 0 236 987, EPA 0 510 356 and WO 95/13090. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Other forms of interferons include interferon beta, gamma, tau and omega, such as Rebif (Interferon beta 1a) by Serono, Omniferon (natural interferon) by Viragen, or Omega Interferon by Boehringer Ingelheim. Oral interferons such as oral interferon alpha by Amarillo Biosciences.

Additional examples of interferons that may be used include pegylated interferon alpha, for example pegylated interferon α-2a, pegylated interferon α-2b, pegylated consensus interferon or pegylated purified interferon-α product. Pegylated interferon α-2a is described in European Patent 593,868 (incorporated herein by reference in its entirety) and commercially available e. g. under the trade name PEGASYS® (Hoffmann-La Roche). Pegylated interferon-α-2b is described, e.g. in European Patent 975,369 (incorporated herein by reference in its entirety) and commercially available e.g. under the trade name PEG-INTRON A® (Schering Plough). Pegylated consensus interferon is described in WO 96/11953 (incorporated herein by reference in its entirety).

In preferred embodiments, the interferon used in the methods of the invention is pegylated interferon. In other embodiments, the interferon is selected from the group consisting of interferon alpha-2a, Interferon alpha-2b, a consensus interferon, a purified interferon alpha product or a pegylated interferon alpha-2a, pegylated interferon alpha-2b, and pegylated consensus interferon, a mixture of natural alpha and combinations thereof.

Preferably the methods using interferon alpha use a pegylated interferon alpha-2b and the amount of pegylated interferon alpha-2b is from 0.5 to 2.0 micrograms/kilogram per week on a weekly, three times a week, every other day or daily basis.

As used herein, “microgram/kilogram” means microgram drug per kilogram body weight of the mammal—including man—to be treated.

As used herein, the term “treatment” or “treat” refer to both prophylactic or preventative treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

By “therapeutic regimen” is meant the pattern of treatment of an illness, e.g., the pattern of dosing used during HCV therapy. A therapeutic regimen may include an induction regimen and a maintenance regimen.

The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both.

The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “about”, unless the context dictates otherwise, is used to mean a range of + or −10%.

In other embodiments, the interferon alpha is a pegylated interferon alpha-2a and the amount of pegylated interferon alpha-2a administered is from 20 to 250 micrograms/kilogram per week on a weekly, three times a week, every other day or daily basis. Preferably, the interferon peg-IFNa2a is administered at an amount of 180 micrograms once per week.

In specific embodiments, the exemplary interferon used in the methods herein is interferon selected from the group consisting of Intron-A®; PEG-Intron®; Roferon®; Pegasys®; Berefor®; Sumiferon®; Wellferon®; Infergen®; Alferon®; Viraferon®; Albuferon® (Human Genome Science); Rebif; Omniferon; Omega and combinations thereof.

In some embodiments, the patient may be administered ribavirin or a ribavirin derivative (e.g., a ribavirin analog or prodrug, such as ribamidine, taribavirin (viramidine), ICN 17261, molecules disclosed in WO/2008/052722, which is incorporated by reference in its entirety, etc.).

In some embodiments, ribavirin is administered at between about 800 mg to about 1200 mg per day, e.g., 800 mg, 900 mg, 1000 mg, 1100 mg, 1200 mg per day. In some embodiments, ribavirin is administered based on the weight of the patient.

In another embodiment, alisporivir may be administered with additional agents of the standard of care that promote the antiviral efficacy of the therapy treatment. The standard of care may include additional agents that promote the antiviral efficacy of the therapy treatment, such as substrate-based protease inhibitors of HCV NS3-4A serine protease, non-substrate-based NS3 protease inhibitors; phenanthrenequinones, thiazolidines and benzanilides, nucleosides analogs, antisense molecules directed against HCV genome or any cellular component that is required for viral replication, vaccine or antibody-based approaches to HCV treatment. Direct acting antiviral agents, is used herein to mean agents that interfere with specific steps in the hepatitis C virus (HCV) replication cycle. Such agents may be, e.g., ribavirin derivatives, protease inhibitors, polymerase inhibitors (e.g., nucleoside and non-nucleoside inhibitors), and cyclophillin inhibitors. Exemplary antiviral include: boceprevir, telaprevir, ABT-072, ABT-450, ABT-333 by Abbott, ACH1625 by Achillion, ANA598 by Anadys Pharmaceuticals, AZD-7295 by AstraZeneca, BI201335, BI207127 by Boehringer Ingelheim Pharma, BMS650032, BMS790052, BMS791325, BMS824383 by Bristol Myers Squibb, Clemizole by Eiger BioPharmacetucials, Filibuvir by Pfizer, GS9190 (Tegobuvir), GS9256 by Gilead, IDX375 by Idenix, INX-189 by Inhibitex, PSI-7851, PSI-938 by Pharmasset, PSI-7977, RG7128 by Pharmasset/Genethec, PPI-461 by Presidio RG7227 (Danoprevir) by InterMune/Genentech, SCH900518 (Narlaprevir), Vaniprevir by Merck, TMC435 by Medivir/Tibotec, VX-222, VX-759, VX-500, VX-916 by Vertex.

In some embodiments, alisporivir may be administered once per day (daily), twice per day, three times per day, every other day, every three days, weekly (once per week), once every other week, once every three weeks, once monthly, etc.

In one embodiment, the present invention further provides alisporivir for use in combination with standard of care in treatment of a Hepatitis C virus infected patient, the alisporivir to be administered in an amount of about 400 to about 600 mg (e.g., about 350 mg, about 400 mg, about 450 mg, about 500 mg, about 550 mg, about 600 mg, about 650 mg) twice per day.

As used herein “twice per day” means twice in any period of about 24 hour period.

As used herein “once per week” is used to mean once in any period of about seven days.

In still another aspect, alisporivir is to be administered for up to 24, 48 or 72 weeks. As used herein “up to 24, 48 or 72 weeks” is used to mean that alisporivir is administered on a continuous basis (e.g., twice per day, once per week, etc.) for about 24 weeks, about 48 weeks, or about 72 weeks. It will be understood that therapy need not end at exactly the 24, 48 or 72 week time period. For example, therapy may end a day or a few days before the 24 week period, and still be an equivalent within the scope and spirit of the current disclosure.

In one embodiment, the present invention further provides alisporivir for use in combination with standard of care in treatment of a chronic Hepatitis C virus genotype 1 infection patient with IL28B nonC/C genotype, the alisporivir being administered during an initial phase in an amount of about 600 mg, twice per day; followed by administering alisporivir during a second phase in an amount of about 600 to about 800 mg once per day. In still another aspect, the initial phase is a period of 7 days; the second phase is a period of 23, 47 or 71 weeks.

In one embodiment, the present invention further provides alisporivir for use in combination with an interferon (e.g., pegylated interferon alpha 2a or pegylated interferon alpha 2b) and ribavirin in treatment of chronic Hepatitis C virus genotype 1 infection in a patient with IL28B nonC/C genotype, the alisporivir being administered during an initial phase in an amount of about 600 mg, twice per day for 7 days; followed by administering alisporivir during a second phase in an amount of about 600 or about 800 mg once per day for up to 23, 47 or 71 weeks.

In one embodiment, the present invention further provides alisporivir for use in combination with interferon and ribavirin in treatment of chronic Hepatitis C virus genotype 1 infection in a patient with IL28B nonC/C genotype, the alisporivir being administered during an initial phase in an amount of about 600 mg, twice per day for 7 days; followed by administering alisporivir during a second phase in an amount of about 600 mg once per day for up to 71 weeks, preferably up to 23 weeks, most preferred up to 47 weeks.

In one embodiment, the present invention further provides alisporivir for use in combination with interferon and ribavirin in treatment of chronic Hepatitis C virus genotype 1 infection in a patient with IL28B nonC/C genotype, the alisporivir being administered during an initial phase in an amount of about 600 mg, twice per day for 7 days; followed by administering alisporivir during a second phase in an amount of about 800 mg once per day for up to 47 weeks.

In one embodiment, the present invention further provides alisporivir for use in combination with standard of care, preferably with pegylated interferon alpha-2a and ribavirin in treatment of chronic Hepatitis C virus genotype 1 infection in a patient with IL28B nonC/C genotype, the alisporivir being is administered during an initial phase in an amount of about 600 mg, twice per day; followed by administering alisporivir during a second phase in an amount of about 600 to about 800 mg once per day for up to 23 or 47 or 71 weeks. In still another aspect, the pegylated interferon alpha-2a and is administered in an amount of about 180 micrograms (e.g 180 micrograms) once per week.

In one embodiment, the present invention further provides alisporivir for use in combination with pegylated interferon alpha-2a and ribavirin in treatment of chronic Hepatitis C virus genotype 1 infection in a patient with IL28B nonC/C genotype, the alisporivir being administered during an initial phase in an amount of about 600 mg, twice per day; followed by administering alisporivir during a second phase in an amount of about 600 to about 800 mg once per day for up to 23, 47 or 71 weeks. In still another aspect, the ribavirin is administered at between about 1000 mg to about 1200 mg per day and the pegylated interferon alpha-2a is administered in an amount of about 180 micrograms once per week.

In one aspect, the present invention further provides a method of treating of chronic Hepatitis C virus genotype 1 infection in a patient with IL28B nonC/C genotype with alisporivir in combination with standard of care, preferably with an interferon and ribavirin, the method comprising administering alisporivir during an initial phase in an amount of about 600 mg, twice per day; followed by administering alisporivir during a second phase in an amount of about 600 to about 800 mg once per day for up to 23, 47 or 71 weeks. In still other aspects, the initial phase is a period of at least 3 days, preferably of 5 days, most preferred of 7 days.

In one aspect, the present invention further provides use of alisporivir in the manufacture of a medicament for treatment of chronic Hepatitis C virus genotype 1 infection in a patient with IL28B nonC/C genotype wherein alisporivir is administered during an initial phase in an amount of about 600 mg, twice per day; followed by administering alisporivir during a second phase in an amount of about 600 to about 800 mg once per day for up 23, 47 or 71 weeks and wherein alisporivir is administered in combination with an interferon and ribavirin throughout the initial and second phases. In still other aspects, the initial phase a period of at least 3 days, preferably of 5 days, most preferred of 7 days.

In one aspect, the present invention further provides use of alisporivir in the manufacture of a medicament for treatment of chronic Hepatitis C virus genotype 1 infection in a patient with IL28B C/C genotype wherein alisporivir is administered during an initial phase in an amount of about 600 mg, twice per day; followed by administering alisporivir during a second phase in an amount of about 600 to about 800 mg once per day for up 23, 47 or 71 weeks and wherein alisporivir is administered in combination with an interferon and ribavirin throughout the initial and second phases. In still other aspects, the initial phase a period of at least 3 days, preferably of 5 days, most preferred of 7 days.

In one aspect, the present invention further provides use of alisporivir in the preparation of a pharmaceutical composition for treatment of chronic Hepatitis C virus genotype 1 infection in a patient with IL28B nonC/C genotype characterized in that alisporivir is administered during an initial phase in an amount of about 600 mg, twice per day; followed by administering alisporivir during a second phase in an amount of about 600 to about 800 mg once per day for up to 23, 47 or 71 weeks and wherein alisporivir is administered in combination with an interferon and ribavirin throughout the initial and second phases. In still another aspects, the initial phase is a period of at least 3 days, preferably of 5 days, most preferred of 7 days.

In one aspect, the present invention further provides use of alisporivir in the preparation of a pharmaceutical composition for treatment of chronic Hepatitis C virus genotype 1 infection in a patient with IL28B C/C genotype characterized in that alisporivir is administered during an initial phase in an amount of about 600 mg, twice per day; followed by administering alisporivir during a second phase in an amount of about 600 to about 800 mg once per day for up to 23 weeks and wherein alisporivir is administered in combination with an interferon and ribavirin throughout the initial and second phases. In still another aspects, the initial phase is a period of at least 3 days, preferably of 5 days, most preferred of 7 days.

In one aspect, the present invention further provides a combination of alisporivir with standard of care, preferably with interferon and ribavirin for use in treatment of chronic Hepatitis C virus genotype 1 infection in a patient with IL28B nonC/C genotype, wherein alisporivir is administered during an initial phase in an amount of about 600 mg, twice per day for 7 days; followed by administering alisporivir during a second phase in an amount of about 600 to about 800 mg once per day for up to 23, 47 or 71 weeks.

In one aspect, the present invention further provides a therapeutic regimen comprising administering alisporivir during an initial phase in an amount of about 600 mg, twice per day for one week; followed by administering alisporivir during a second phase in an amount of about 600 to about 800 mg once per day for up to 23, 47 or 71 weeks and wherein alisporivir is administered in combination with interferon and ribavirin throughout the initial and second phases.

In one aspect, the present invention further provides pharmaceutical compositions comprising alisporivir for uses as defined above. In still other aspects, the present invention provides a package comprising the pharmaceutical composition comprising alisporivir for uses as defined above in combination with instructions to administer said composition.

In still other aspects, the present invention provides a package comprising instructions to administer alisporivir according to any method described herein.

In exemplary embodiments, alisporivir is administered at a dosage of from about 600 to about 1000 mg twice daily for 7 days followed by administering alisporivir at a dosage of from about 600 to about 1000 mg once per day for up to 23, 47 or 71 weeks.

In exemplary embodiments, the treatment of the present invention involves administration of interferon alpha that is a pegylated interferon alpha-2a and the amount of pegylated interferon alpha-2a administered is from 20 to 250 micrograms per week on a weekly, three times a week, every other day or daily basis. The current approved dose is 180 micrograms per week. In other exemplary embodiments, the interferon alpha is a pegylated interferon alpha-2b and the amount of pegylated interferon alpha-2b is from 0.5 to 2.0 micrograms/kilogram per week on a weekly, three times a week, every other day or daily basis. Exemplary descriptions of such treatments are described in U.S. Pat. No. 7,115,578, incorporated herein by reference in its entirety.

An exemplary Peg-IFNα2a used in the treatment protocols described herein is Pegasys®. PEGASYS® is a pegylated form of IFNα2a and utilizes a 40 kDa branched PEG (polyethylene glycol) to provide sustained serum concentrations for a full week (168 hours). PEGASYS® is commercially available, presented as single use, pre-filled syringes containing 180 μg/0.5 mL peg-IFNα2a for S.C. injection. The standard package contains 1 syringe of 180 μg/0.5 mL.

In some embodiments, it may be desirable to modify the dose of Peg-IFNα2a. If dose modification is required for moderate to severe adverse reactions (clinical and/or laboratory), initial dose reduction from 180 to 135 μg is generally adequate (adjustment to the corresponding graduation mark on pre-filled syringe). However, in some cases, dose reduction to 90 μg may be needed. Following improvement, re-escalation of the dose may be considered.

In treatment described above effective dosages of the standard of care agents are administered in compositions, i.e. they may be administered together (i.e., simultaneously), but may also be administered separately or sequentially. In general, combination therapy is typically administered together, the rationale being that such simultaneous administration induces multiple simultaneous stresses on the virus. The specific dosages given will depend on absorption, inactivation and excretion rate of the drugs as well as other factors. It is to be noted that dosage values will also vary with the severity of the condition to be alleviated.

The terms “co-administration” or “combined administration” or “administered in combination with” or the like as utilized herein are meant to encompass administration of the selected therapeutic agents to a single patient, and are intended to include treatment regimens in which the agents are not necessarily administered by the same route of administration or at the same time. Fixed combinations are also within the scope of the present invention. The administration of a pharmaceutical combination of the invention results in a beneficial effect, e.g. a synergistic or additive therapeutic effect, compared to a monotherapy applying only one of its pharmaceutically active ingredients or as compared to the current standard of care therapy. The treatment used in the methods described herein may be administered by any conventional route. One or more components may be administered parentally, e.g., in the form of injectable solutions or suspensions, or in the form of injectable deposit formulations. Preferably, alisporivir will be administered orally in the form of solutions or suspensions for drinking, tablets or capsules. Pharmaceutical compositions for oral administration comprising alisporivir typically further comprise one or more pharmaceutically acceptable carrier substances. Typically, these compositions are concentrated and need to be combined with an appropriate diluent, e.g., water, prior to administration. Pharmaceutical compositions for parenteral administration typically also include one or more excipients. Optional excipients include an isotonic agent, a buffer or other pH-controlling agent, and a preservative. These excipients may be added for maintenance of the composition and for the attainment of preferred ranges of pH (about 6.5-7.5) and osmolarity (about 300 mosm/L).

The efficacy of the therapy regimen may be monitored using standard protocols. Treatment may be followed by determinations of HCV in serum and measurement of serum ALT levels. For example, the patients may be assessed for the presence of HCV RNA in their plasma. HCV RNA (IU/mL) can be measured at regular intervals during the treatment, e.g., at Day 1 (pre-dose and 4, 8, and 12 hours post-dose) and pre-dose at Day 2, Day 3, Day 8, Day 15, Day 29, and at Week 12, Week 24, Week 36, Week 48, Week 72 (when applicable), and at follow up. In addition, the HCV strains in the patient can be sequenced and assessed for identification of mutations selecting for resistance.

The endpoint of treatment is a virological response, i.e., the absence of HCV at the end of a treatment course, several months after initiation of treatment, or several months after completion of treatment. HCV in serum may be measured at the RNA level by methods such as quantitative RT-PCR or northern blots or at the protein level by enzyme immunoassay or enhanced chemiluminescence immunoassay of viral proteins. The endpoint may also include a determination of a serum ALT level in the normal range.

Exemplary therapeutic regimes are given in the Examples.

The following Examples illustrate the invention described hereinbefore.

### EXAMPLES

In one exemplary regimen a subject in need of treatment is provided with pegylated interferon alfa 2a (peg-IFNα2a) at a dose of 180 μg subcutaneously (S.C.) once weekly for 48 weeks in combination with ribavirin administered in an oral dosage of 1000/1200 mg daily (weight based) for 48 weeks and 600 mg alisporivir orally twice daily for 7 days, followed by 600 to 800 mg alisporivir orally once daily for 47 weeks.

In another exemplary protocol a subject in need of treatment is provided with pegylated interferon alfa 2a at a dose of 180 μg subcutaneously (S.C.) once weekly for 48 weeks in combination with ribavirin administered in an oral dosage of 1000/1200 mg daily (weight based) for 48 weeks and 600 mg alisporivir orally twice daily for 7 days, followed by 800 mg alisporivir orally once daily for 47 weeks.

After a 4 week treatment period, based on patient response, the administration of alisporivir may be continued up to 48 or 72 weeks from the start of treatment at 600 or 800 mg per day orally or preferably the dose of alisporivir is reduced to a lesser amount in a daily dose (e.g., 400 or 600 mg) or more preferably, the administration of alisporivir is discontinued. The treatment with pegylated interferon alfa 2a and ribavirin is preferably continued for up to 48 or 72 weeks from the initiation of treatment. For example between weeks 5 to 48 or 72, the patient is administered 180 μg pegylated interferon alfa 2a S.C. orally once weekly and ribavirin administered in an oral dosage of 1000/1200 mg daily (weight based).

### 1. Compounds

Peg-IFNα2a is a pegylated form of interferon alfa 2a and utilizes 40 kDa branched PEG (polyethylene glycol) to provide sustained serum concentrations for a full week (168 hours). PEGASYS® is commercially available from Roche.

Ribavirin is a synthetic nucleoside analogue and is also commercially available, e.g., as COPEGUS® from Roche.

### 2. Clinical Study and Results

An international, multicentre, randomised, double-blind, placebo-controlled, 4-arm parallel-group multiple dose study comparing three alisporivir/peg-IFNα2a/ribavirin regimens to standard of care treatment in 272 treatment naïve chronic HCV genotype 1 patients is provided.

Patients are randomized into one of 3 treatment arms (A, B and C) as described below in a 1:1:1 ratio. The randomization is stratified by baseline HCV RNA (HCV RNA<800000 IU/mL or HCV RNA≧800000 IU/mL) and BMI (BMI<25 kg/m2 or BMI≧25 kg/m2) at screening.

### Treatment A

Alisporivir: 3 capsules of 200 mg (600 mg) alisporivir 2×/day orally for 1 week followed by 3 capsules of 200 mg alisporivir (600 mg) 1×/day for 47 weeks.

Pegylated interferon alfa 2a: 180 μg subcutaneously (s.c.) once weekly for 48 weeks

Ribavirin: 1000 mg/day (<75 kg) or 1200 mg/day (≧75 kg) orally in two divided doses for 48 weeks

### Treatment B

Alisporivir: 3 capsules of 200 mg (600 mg) alisporivir 2×/day orally for 1 week followed by 3 capsules alisporivir (600 mg) 1×/day for 23 weeks.

Pegylated interferon alfa 2a: 180 μg subcutaneously (s.c.) once weekly for 48 weeks

Ribavirin: 1000 mg/day (<75 kg) or 1200 mg/day (≧75 kg) orally in two divided doses for 48 weeks

Recent evidence shows that adding a third drug to SOC treatment improves SVR even when treatment duration is reduced, especially if the triple combination induces a more rapid decline in viral load and, as a result, a higher percentage of rapid viral response (RVR) (see Flisiak, R., et al. The cyclophilin inhibitor DEBIO-025 has a potent dual anti-HIV and anti-HCV activity in treatment-naïve HIV/HCV co-infected subjects. in 57th annual Meeting of the American Association for the Study of Liver Diseases (AASLD) (poster). 2006. Boston, USA).

Reduced total treatment duration could improve the safety profile by reducing the incidence of late onset adverse effects, could improve treatment adherence—an important factor in the success rate—and would certainly diminish treatment costs. Recent experience with protease inhibitors indicates that a 24-week treatment with a triple combination therapy (peg-IFNα2a/ribavirin/telaprevir) is sufficient to obtain a higher proportion of SVR patients than standard of care treatment for 48 weeks (61 vs. 41%) (see McHutchison, J. G., et al., PROVE1: Results from a phase 2 study of telaprevir with peginterferon alfa-2a and ribavirin in treatment-naïve subjects with hepatitis C. J Hepatol, 2008. Suppl 2, Vol 48 p. S4)

### Treatment C

Alisporivir: 3 capsules of 200 mg (600 mg) alisporivir 2×/day orally for 1 week followed by 3 capsules alisporivir (600 mg) 1×/day for 23 weeks or for 47 weeks.

Pegylated interferon alfa 2a: 180 μg subcutaneously (s.c.) once weekly for 48 weeks

Ribavirin: 1000 mg/day (<75 kg) or 1200 mg/day (≧75 kg) orally in two divided doses for 48 weeksExperience with IFN-based therapies indicates that it may be possible to optimise treatment results by tailoring treatment duration to viral response (see Zeuzem, S., et al., Efficacy of 24 weeks treatment with peginterferon alfa-2b plus ribavirin in patients with chronic hepatitis C infected with genotype 1 and low pretreatment viremia. J Hepatol, 2006. 44(1): p. 97-103). Therefore, this arm will use a flexible treatment regimen of 24 weeks for patients who achieve RVR and 48 weeks for patients who do not achieve RVR. The aim of treating non-RVR patients for 48 weeks is to maximally reduce the proportion of patients who relapse after treatment.

Patients who achieve RVR (HCV RNA<10 IU/mL at week 4) will continue treatment for a total duration of 24 weeks:
Peg-IFNα2a 180 μg s.c. once weekly for 20 weeks
Ribavirin 1000 or 1200 mg/day orally (weight based) for 20 weeks
Alisporivir 600 mg orally once daily for 20 weeks

Patients who do not achieve RVR (HCV RNA≧10 IU/mL at week 4) will continue treatment for a total duration of 48 weeks.
Peg-IFNα2a 180 μg s.c. once weekly for 44 weeks
Ribavirin 1000 or 1200 mg/day orally (weight based) for 44 weeks
Alisporivir 600 mg orally once daily for 44 weeks

### Treatment D

Alisporivir placebo: 3 capsules of placebo (to alisporivir 200 mg) 2×/day orally for 1 week followed by 3 capsules (3×200 mg) placebo 1×/day for 47 weeks.

Pegylated interferon alfa 2a: 180 μg subcutaneously (s.c.) once weekly for 48 weeks

Ribavirin: 1000 mg/day (<75 kg) or 1200 mg/day (≧75 kg) orally in two divided doses for 48 weeks

Primary efficacy endpoint: proportion of patients achieving SVR 24 (HCV RNA<10 IU/mL at maximum 24 weeks after treatment end).

### 3. Genotyping Analysis

Genotyping of IL-28B by was carried out using the MALDI-TOF MS (matrix-assisted laser desorption/ionization time-of-flight mass spectrometer) technique which has emerged as a widely applicable method in molecular biology for fast and highly accurate DNA analysis. All reactions within this study followed the protocols defined in the iPLEX® Gold Application Guide.

Frequency of genetic polymorphisms of IL28B was calculated by treatment arm. Pearson's chi-square test or Fisher exact test were used to analyse the relationship between SVR at Week 72 and the genotype classes.

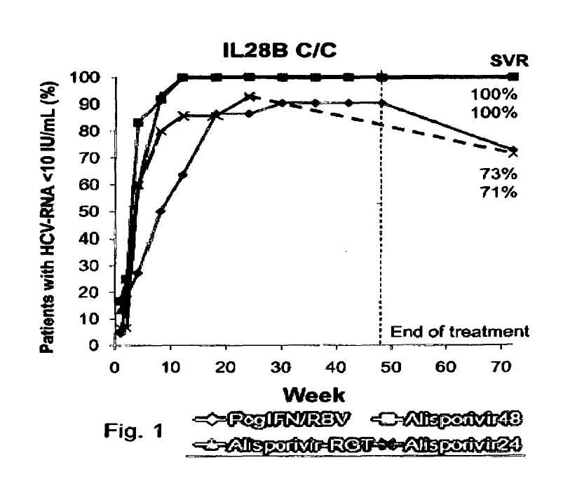

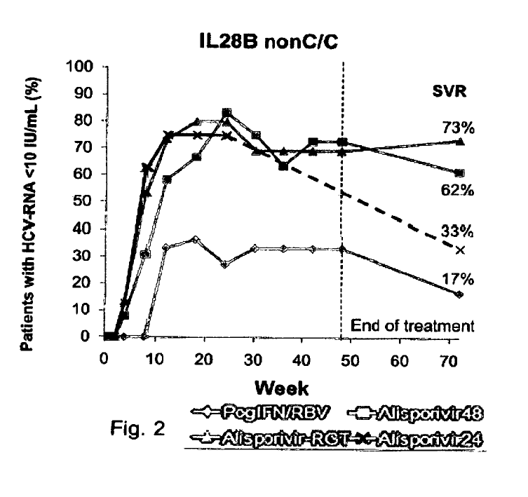

Strong associations were observed between IL28B polymorphisms and the viral responses and the viral load reduction starting from week 1 of treatment. For the IL28B rs12979860 SNP, individuals harboring the minor T allele showed a significantly smaller chance of achieving SVR24. The results are shown in FIG. 1 and FIG. 2.

1. A method of treating of chronic Hepatitis C virus genotype 1 infection in a patient with IL28B nonC/C genotype comprising administering alisporivir to the patient during an initial phase in an amount of about 600 mg, twice per day for 7 days; followed by administering alisporivir during a second phase in an amount of about 600 to about 800 mg once per day for up to 23, 47 or 71 weeks and optionally administering the patient standard of care or a direct antiviral agent.

2. A method of treating of chronic Hepatitis C virus genotype 1 infection in a patient comprising
(i) determining the with IL28B genotype
(ii) administering alisporivir to the patient during an initial phase in an amount of about 600 mg, twice per day for 7 days; followed by administering alisporivir during a second phase in an amount of about 600 to about 800 mg once per day for up to 23, 47 or 71 weeks and optionally administering the patient standard of care or a direct antiviral agent to a patient with IL28B nonC/C genotype
(iii) administering alisporivir during an initial phase in an amount of about 400 to about 600 mg, twice per day followed by administering alisporivir during a second phase in an amount of about 600 to about 800 mg once per day and wherein alisporivir is administered in combination with standard of care throughout the initial and second phases to a patient with IL28B C/C genotype and wherein the treatment duration is halved compared to the treatment duration of the approved standard of care treatment duration for genotype 1 patients.

3. A combination of alisporivir, and standard of care for use in treatment of chronic Hepatitis C virus genotype 1 infection in a patient with IL28B nonC/C genotype characterized in that alisporivir is administered during an initial phase in an amount of about 600 mg, twice per day for 7 days; followed by administering alisporivir during a second phase in an amount of about 600 to about 800 mg once per day for up to 23, 47 or 71 weeks.

4. A pharmaceutical composition comprising alisporivir for use according to claim 1.

5. A package comprising the pharmaceutical composition according to claim 4 in combination with instructions to administer said composition.
